(19) United States
(12) Patent Application Publication
(10) Pub. No.: US 2003/0236397 A1
(43) Pub. Date: Dec. 25, 2003

(54) Process for preparing beta-L-2'deoxy-thymidine

(75) Inventors: Franz Dietrich Klingler, Griesheim (DE); Paul Kreye, Ingelheim (DE); Wolfgang Baumgarten, Gau-Algesheim (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/413,020

(22) Filed: Apr. 14, 2003

(30) Foreign Application Priority Data
Apr. 12, 2002 (DE) DE 102 16 426.6

(51) Int. Cl. C07H 19/073

(52) U.S. Cl. 536/28.53

(57) ABSTRACT

The present invention relates to a new, essentially four-step process for preparing beta-L-2′-deoxy-thymidine starting from L-arabinose. The process according to the invention is particularly important for mass production of beta-L-2′-deoxy-thymidine.

### FIELD OF THE INVENTION

The present invention relates to a new, four-step process for preparing beta-L-2′-deoxy-thymidine starting from L-arabinose. The process according to the invention is of particular importance for the mass production of beta-L-2′-deoxy-thymidine.

### BACKGROUND OF THE INVENTION

Beta-L-2′-deoxy-thymidine, which does not occur naturally and is abbreviated to LdT within the scope of the present invention, is a nucleoside analogue of great pharmacological interest and as such is of great importance for the production of medicaments, particularly antiviral medicaments. Thus, for example, LdT has proved helpful in the fight against HIV-associated diseases. In terms of its chemical structure LdT is represented by general formula I:

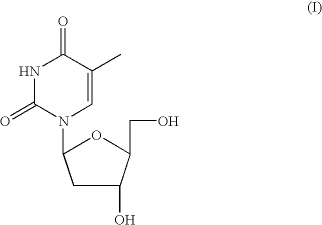

Naturally occurring thymidine, which is not directly a subject of the present invention, is the addition product of thymine and deoxyribose and as such is an important component of DNA. As a component of the carrier of inherited information thymidine is produced in the living body by the methylation of uridine.

However, the present invention does not relate to the naturally occurring thymidine, but to its enantiomer, namely beta-L-2′-deoxy-thymidine, which is formally produced by exchanging the D-2-deoxy-ribose in natural 2-deoxy-thymidine for L-2-deoxyribose or L-2-deoxyarabinose.

Numerous attempts at chemically synthesising naturally occurring D-2′-deoxy-thymidine are known from the prior art. Suggestions of mass production are also found therein.

One of the original methods of producing pyrimidine-nucleosides was proposed in 1972 by A. Holý (Coll. Czech. Chem.Com. 37, 4072 (1972)). This article described, among other things, precursor molecules for D-arabino-pyrimidines-nucleoside analogues. From the range of molecules derived from thymine the preparation of beta-D-2,2′-anhydro-arabinofuranosyl-thymine from D-arabinose is disclosed.

In Tetrahedron Letters 38 (40), 7025-7028, (1997) a stereospecific synthesis of D-2′-deoxy-ribofuranosyl-pyrimidines is described in which D-ribose is reacted in a first step with cyanamide and then with alpha-methyl-glycidate. The thus obtained beta-D-2,2′-anhydro-uridine derivative is then converted in beta-D-2′-deoxy-ribofuranosyl-thymine through a number of steps.

U.S. Pat. No. 4,914,233 discloses a process for preparing beta-2-deoxy-thymidine starting from D-ribose, which is first reacted to form tri-O-acetyl-thymidine.

In addition to methods of this kind for preparing the naturally occurring nucleosides there are also suggestions on the synthesis of the L-analogues.

For example WO 01/34618 proposes a complicated, multi-step process for LdT, which starts from L-arabinose and passes through a thio intermediate.

WO 96/13512 describes the synthesis of beta-L-2′-deoxy-uridine, which has to be converted into LdT in subsequent steps.

A similarly complicated approach is described in WO 92/08727. Again a method of synthesis is proposed in which finally beta-L-2′-deoxy-uridine is reacted to form LdT

Other methods of preparing LdT from beta-L-2′-deoxy-uridine are also described in WO 00/09531, for example.

It is an aim of the present invention to provide a new process for preparing LdT which overcomes the disadvantages of the processes known from the prior art.

Another aim of the present invention is to develop a process with as few reaction steps as possible.

Yet another aim is to develop a process for LdT in which there is no need for any laborious separation of an alpha-beta mixture.

The present invention also sets out to develop a large-scale industrial process for LdT.

Yet another aim is to develop a large-scale industrial process for LdT in which the use of chlorinated solvent can be reduced or eliminated entirely.

Yet another aim is to develop an economical process for producing LdT.

### DETAILED DESCRIPTION OF THE INVENTION

The process according to the invention achieves these objectives by a process comprising four key steps.

In a first step L-arabinose is reacted with cyanamide to form an amino-oxazoline derivative. The oxazoline ring is synthesised from the cyanamide, the anomeric carbon atom and the oxygen in the 2 position of the arabinose. The other hydroxy groups of the intermediate may optionally be blocked with conventional protective groups, with or without working up of the reaction mixture, to prevent additional unwanted reactions. Common protective groups are described for example in Greene et al., Protective groups in Organic Synthesis, John Wiley and Sons, Second edition 1991 or another edition thereof. We refer explicitly to the corresponding chapter of this monograph (“OH protective groups”) in this context.

The oxazoline derivative formed in the first step is represented by general formula II:

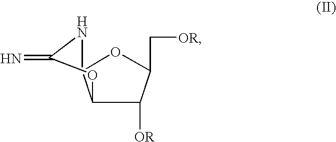

where R denotes hydrogen or a protective group as defined elsewhere in this specification.

In a second step the reaction product of the first step is reacted with a 2-methyl-C-3-acid or derivative thereof, e.g. an activated derivative thereof, to obtain beta-L-2,2′-anhydro-thymidine according to general formula III:

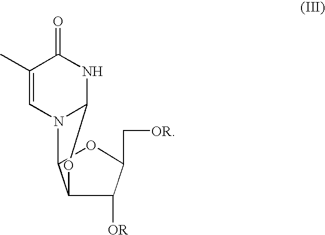

The group R denotes hydrogen or a protective group, as defined elsewhere in this specification.

This intermediate according to formula III is then converted in a third step into an L-thymidine derivative with a reducible carbon in 2′ position of general formula IV and finally reduced to the LdT in the fourth reaction step. Formula IV:

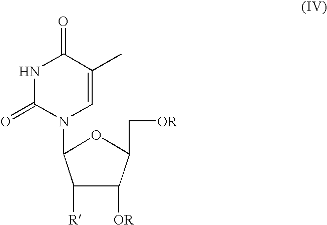

The reaction steps are shown in Diagram 1 on the next page as an illustration.

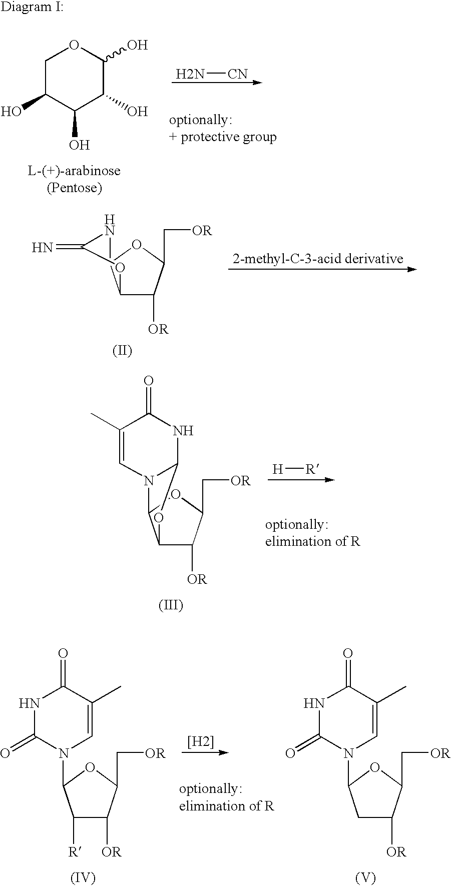

Here the term protective group denotes a hydroxy protective group, e.g. as described in the abovementioned monograph by Greene et al.. The purpose of the protective group is particularly to prevent the free OH groups of the oxazoline derivative from reacting with the 2-methyl-C-3-acid in the second reaction step.

Preferably, the protective groups are those which can be cleaved under acidic conditions or reductive conditions. Protective groups of this kind have the advantage that they are cleaved under the reaction conditions of the third or fourth reaction step.

Preferred protective groups are benzyl, diphenylmethyl, triphenylmethyl or silyl protective groups, the three substituents of the silyl being selected from among the C₁-C₆-alkyls and/or phenyl. The phenyl groups of all the abovementioned protective groups may optionally be substituted, for example, with C₁-C₆-alkyl; nitro and/or C₁-C₆-alkoxy. Preferred protective groups are the trimethylsilyl, dimethyl-tert.butyl-silyl, diphenyl-tert.butyl-silyl and tributylsilyl protective groups.

However, it is also possible to use other protective groups as well, which are cleaved in another reaction step.

Although it is preferable to protect the OH groups, it is not essential. Therefore the group R in the formula element “OR” does not represent the corresponding moiety of the protective group, but also denotes hydrogen. In other words: R in the term “OR” preferably denotes hydrogen, benzyl, diphenylmethyl, triphenylmethyl, or silyl, the three substituents of which are selected from among the C₁-C₆-alkyls and/or phenyl. The phenyl groups in all the variants mentioned may optionally be substituted, for example with C₁-C₆-alkyl, nitro and/or C₁-C₆-alkoxy.

The 2-methyl-C-3-acid or the derivative thereof is preferably selected from the group methyl-2-formyl-propionate, another 2-formyl-propionic acid ester, 2-formyl-propionitrile, -azide or -halide, the dimethoxy or diethoxy-acetal of the abovementioned formyl compounds, a 3-z-2-methyl-2-propenoic acid ester, azide, halide or nitrile, while z is selected from among F, Cl, Br, I, O-tosylate, or C₁-C₆-alkoxy, such as methoxy, ethoxy etc. The esters mentioned are preferably the methyl, ethyl, propyl and butyl esters. Instead of the acid derivatives it is also possible to use the corresponding acids or activated acids. The acid may be activated using the activating reagents and acid adducts known from peptide coupling, for example. At this point we refer to the relevant specialist literature.

R′ denotes Cl, Br, I, tosyl or thioacetyl, preferably Cl, Br, I and most preferably Br.

In the first reaction step L-arabinose is reacted with cyanamide. The reaction may take place in an aqueous, aqueous-alcoholic (e.g. methanol) or other polar solvent. Suitable solvents include water-methanol mixtures, dimethylformamide (DMF), pyridine, N-methylpyrrolidone (NMP) etc. The reaction is preferably carried out at high temperatures, preferably between 50° C. and the boiling point of the corresponding solvent, more preferably between 70° C. and 120° C., most preferably between 80° C. and 100° C. The presence of a base catalyses the reaction. Suitable bases include, for example, ammonia, tertiary amines such as triethylamine or carbonate. Alternative reaction conditions known in the art may be used.

Optionally, this step may be followed by protection of the remaining OH groups of the L-arabinose. It is not absolutely necessary to work up the reaction mixture of the first step completely beforehand. As a rule it is sufficient if the corresponding pH value of the solvent is adjusted and then the reagents needed to protect the OH groups are added. As water generally interferes with the reaction of alcoholic hydroxy groups with the corresponding protective groups, the first reaction step (cyanamide coupling) in this case is preferably carried out in an anhydrous medium such as DMF, pyridine or NMP. Preferably, DMF is used. As reactive bases such as ammonia can also interfere with this step, tertiary organic nitrogen bases or inorganic bases such as carbonate are preferably used as bases for the cyanamide coupling. Alkali metal or dialkali metal carbonates are most preferred.

Preferably, the OH groups are protected as silylethers. For this, first of all, remaining carbonate is removed from the reaction mixture of the first step by the addition of an acid such as sulphuric acid, for example. Then the reaction conditions for silylation are created. The reaction conditions may be found in the specialist literature, for example the monograph by Greene et al mentioned earlier.

The oxazolines of general formula II obtained from this reaction step are also a subject of the present invention.

In the second reaction step the oxazoline derivative obtained by the first reaction step is reacted with the 2-methyl-C-3-acid or a derivative thereof. Preferably, methyl-2-formylpropionate is used as the 2-methyl-C-3-acid or a derivative thereof. The reaction takes place in an inert solvent under water-separating conditions, for example a C₁- to C₄-alcohol, dimethylsulphoxide, DMF, NMP, acetone, dimethylacetamide, cyclohexane, benzene, toluene etc. Preferably, no alcohols are used. The water released may either be chemically bound, or it is removed using a water separator, to speed up the reaction.

Catalysts may be added to the reaction, for example tertiary nitrogen bases or inorganic salts. Examples include dimethylaminopyridine, triethylamine, N-methylmorpholine, or mixtures thereof.

The reaction temperature is usually between 0° C. and 150° C., depending on whether the water released is chemically bound or is to be eliminated by distillation. Preferably, the reaction temperature is 20° C. to 80° C. (or the boiling point of the solvent used).

The beta-L-2,2′-anhydro-thymidine produced by the reaction or the OH-protected derivative thereof is also a subject of the invention.

In the third reaction step the anhydro compound of the second reaction step is reacted with a nucleophile in order to break the C—O bond of the carbon atom in the 2′-position to obtain the oxygen. At the same time the O-group in the 2′-position is exchanged for the nucleophile while reversing the configuration at the carbohydrate carbon. Preferably a halogen (preferably Cl′ Br′ I⁻), tosylate or thioacetate is used as the nucleophile. The reagent used may be the corresponding hydrohalic acid, toluenesulphonic acid, thioacetic acid or a salt thereof. This reaction preferably takes place under acid conditions. Preferably, HCl or HBr is used as the nucleophilic reagent.

Suitable solvents for these reactions include for example DMF or trifluoroacetic acid (TFA).

If an anhydro-thymidine with acid-unstable protective groups (e.g. silyls such as trimethylsilyl or tributylsilyl) for the oxygen atoms in the 3′- and 5′-position is used as the educt for this step, these protective groups are simultaneously removed under the acidic reaction conditions.

In the fourth reaction step the nucleophile introduced by the third reaction step is exchanged for hydrogen under reductive conditions. This reaction takes place under a hydrogen atmosphere, preferably in the presence of a catalyst such as Raney nickel or palladium (e.g. Pd on charcoal). Alternatively the hydrogen may be prepared in situ or a tin hydride such as tributyltin hydride together with a radical starter such as AIBN may be used.

If reductive cleavable but acid-insensitive protective groups were used in the first reaction step, these are now cleaved under the reaction conditions of the fourth step.

If at the start of the process protective groups were introduced which cannot be cleaved either under acid conditions or under reductive conditions, they are now cleaved in an additional reaction step.

At the end of all the reaction steps LdT is obtained. This may optionally be obtained in pure form by crystallisation or other purification steps.

In order that this invention be more fully understood, the following examples of are set forth. These examples are for the purpose of illustrating embodiments of this invention, and are not to be construed as limiting the scope of the invention in any way.

### EXAMPLES

### Example 1

Step 1: 2-amino-beta-D-arabinofuran[1,2′:4,5]-2-oxazolin-di-O-trimethylsilylether

L-(+)-arabinose (30.0 g, 0.20 mol), cyanamide (8.6 g, 0.205 mol), 200 ml DMF and 2.0 g potassium bicarbonate are stirred for 50 min. at 90° C. The mixture is cooled to RT and combined with 0.6 ml of conc. sulphuric acid. This is stirred for 5 min. and then combined with 100 ml of hexamethyldisilazane (0.48 mol) and trimethylsilyl chloride (1.0 ml; 0.008 mol). After about 25 min. a yellow solution is formed which is cooled to 0° C. and combined with 500 ml of toluene. It is extracted with 500 and 200 ml of 10% potassium carbonate solution and the aqueous phases are washed with 100 ml of toluene. The toluene phase is distilled in order to dry it, stirred with 2 g of activated charcoal for 15 min, filtered off and evaporated down again to a total weight of approx. 600 g. 700 ml of hexane are added and the mixture is heated until dissolved (approx. 65° C.). After slow cooling to 0° C. crystallisation begins. The crystals are filtered and washed with toluene/hexane 1:9 and then dried. About 50 g of the desired compound are obtained.

Step 2: Reaction of methyl 2-formylpropionate with the Oxazoline from Step 1

a) Synthesis of methyl 2-formylpropionate

Variant 1:

Methyl methacrylate (6.0 g, 0.06 mol) is cooled to 0° C. and bromine (9.6 g; 0.06 mol) is added dropwise thereto. The reaction temperature should not exceed 20° C. The reaction mixture is stirred for another 2 h (while excluding moisture).

This solution is added to a solution of sodium methoxide (6.5 g; 0.12 mol) in dry methanol (100 ml) and the solution is refluxed for 1 h. The resulting suspension is filtered to separate off the sodium bromide, and evaporated down to the residue. This is taken up in saturated ammonium chloride solution and extracted with MTBE. After drying over magnesium sulphate and evaporation the mixture is distilled in vacuo. The ester is obtained in a yield of 8.9 g (92%) (bp: 101-102° C./42 Torr).

10 g of 15% sulphuric acid are added with stirring to a suspension of 100 g silica gel in 200 ml dichloromethane. After about 3 min., 5 g of 3,3-dimethoxy-2-methylpropionate is added and the mixture is stirred overnight. The reaction mixture is combined with 3,5 g sodium hydrogen carbonate, the solid phase is filtered off and washed. After the solvent has been distilled off 2.9 g (80%) of the product is obtained.

Variant 2:

0.229 l of 2.4M n-BuLi solution in hexane are added dropwise at −78° C. to 90 ml of tetrahydrofuran and 60.72 g (0.6 mol) diisopropylamine. After 30 min stirring 44.50 g (0.50 mol) of methyl propionate are slowly added and stirred for 15 min at −78° C. Then 45.04 g (0.75 mol) of methylformate are added. The yellowish suspension obtained is heated overnight to 0° C. and quenched with 250 ml of 4.4M sulphuric acid. The reaction mixture is extracted with ethyl acetate, the organic phase is dried and finally removed. After distillation 23.88 g methyl-2-formylpropionate is obtained.

b) Reaction with the Oxazoline from Step 1

Variant 1: Boiling in Cyclohexane

A solution of 3.17 g (10 mmol) of the oxazoline (Step 1) in 50 ml cyclohexane is combined with 11.6 g (100 mmol) of methyl 2-formylpropionate and boiled using the water separator. After the reaction has ended, excess cyclohexane is removed and the residue is taken up in 50 ml THF. 20 ml of 1M tetrabutylammonium fluoride solution is added and the resulting mixture is stirred until the silyl protective groups have been cleaved completely. The solvent is concentrated by evaporation and the residue purified by chromatography. 1.6 g (66%) is obtained.

Variant 2:

3,3-dimethoxy-2-methylpropionate (8,1 g; 50 mmol) is dissolved in 100 ml of ice-cold 2N HCl and stirred for 1 h at RT. The solution is cooled to 0° C. and carefully neutralised with 2 N NaOH. This solution is added to an aqueous solution of 15.9 g (50 mmol) of oxazoline (Step 1) and calcium hydroxide (3.2 g). After 24 h stirring at RT the mixture is neutralised with saturated ammonium chloride solution and evaporated down. The solid residue is extracted with hot ethyl acetate. The organic phases are evaporated down and with the addition of hexane (1:1 hexane/chloroform) crystallised. 5.9 g (49%) of the product are obtained.

Step 3: Bromination

2.4 g (10 mmol) of anhydrothymidine from the previous step are dissolved in a solution of 1.0 g HBr in 25 ml DMF and stirred for 40 min at 100° C. The mixture is diluted with 50 ml of ethanol and neutralised with bicarbonate solution. The product can be crystallised by filtering off the inorganic constituents, evaporation and co-distillation with ethanol. recrystallisation from ethanol yields 1.7 g (80%) of the desired product.

Step 4: Hydrogenolysis to Obtain LdT

Variant 1:

The product from Step 4 is taken up in 50 ml of water and hydrogenated at approx. 1.5 bar in the presence of Ra—Ni (50% suspension in water). After the reaction has ended (approx. 2-3 h) the mixture is filtered through Celite. The filter cake is washed with ethanol/water. The combined phases are evaporated down, taken up with octanol and again distilled down by half in vacuo, in order to eliminate DMF and other solvents. After the remaining solvent has been decanted off a residue is left which is boiled with ethyl acetate. The desired product is precipitated out. The residue is filtered off and washed with ethyl acetate. Approx. 1.7 g (80%) of the LdT is obtained.

Variant 2:

Pd/C is used instead of Ra—Ni.

### Example 2

Step 1: 2-amino-beta-D-arabinofuran[1,2′:4,5]-2-oxazolin-di-O-trimethylsilylether

A conc. ammonia solution (50 ml) is combined with 84 g of crystalline cyanamide. The mixture is added with stirring to a mixture of 150 g of L-arabinose in 500 ml of methanol. After 4 h stirring at 45° C. the mixture is poured onto ice water and then filtered, washed and dried. It is then silylated analogously to Example 1, Step 1. A white powder is obtained (181 g).

Step 2: Reaction of methyl 2-formylpropionate with the Oxazoline from Step 1

a) Synthesis of methyl-3-bromomethacrylate

13 ml of bromine are added dropwise to 25.00 g of methyl-methacrylate and the mixture is stirred for 24 h at ambient temperature. The reaction mixture is washed with sodium hydrogen sulphite solution and then extracted with diethylether. The ethereal phase is dried and the ether is eliminated in vacuo. Methyl-2,3-dibromo-2-methylpropionate is obtained as a colourless oil (65 g).

This oil is dissolved in 50 ml of methanol and added to a solution of 9.2 g sodium methoxide in 90 ml of methanol. After 12 hours' stirring the solvent is removed from the solution and the residue is taken up in water and extracted with ethyl acetate. The organic phase is dried, the solvent removed. After distillation, 17 g of a fraction which distils at approx. 69-79° C. is obtained.

b) Reaction with the Oxazoline from Step 1

A suspension of 0.9 g of the products of Step 1, Example 2, 0.90 g of methyl-3-bromomethacrylate, 60 mg of 4-dimethylaminopyridine and 1 ml of triethylamine are heated to 80° C. for 4 days. Then the mixture is diluted with methanol and the solid precipitated is filtered off and discarded. After chromatography 30 mg of an oil are obtained.

Step 3: Bromination

A mixture of 1.5 g of the anhydrothymidine obtained in the second step are stirred in 40 ml with HBr saturated trifluoroacetic acid in a steel bomb at approx. 35° C. for 2 days. Then the solvent is removed in vacuo. The oil remaining is suspended in petroleum ether and the petroleum ether is removed. The residue is recrystallised from ethanol. Colourless crystals are obtained.

Step 4: Hydrogenolysis to Obtain the LdT

110 mg of 2-bromothymidine from the preceding step are combined with 300 mg tributyltin hydride in 5 ml of toluene. After the addition of a spatula tip of AIBN the mixture is refluxed. After 30 min it is cooled, the solvent is removed and the residue is recrystallised from ethyl acetate.

We claim:

1. A process for preparing beta-L-2′deoxy-thymidine according to formula (I): 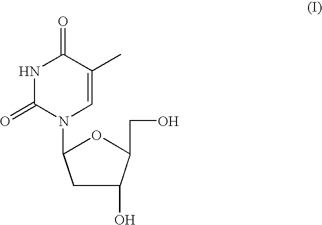
said process comprising the following steps:
a) a first step in which L-arabinose is reacted with cyanamide and then optionally, with or without previous working up of the resulting reaction mixture, the hydroxy groups in the 3- and 5-positions of the L-arabinose are reacted with a protective group to form a compound of formula (II): 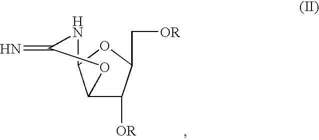
where R is hydrogen or a protective group;
b) a second step in which the product obtained in step a) is reacted with a 2-methyl-C-3-acid or a derivative thereof, to obtain beta-L-2,2′-anhydro-thymidine according to formula (III): 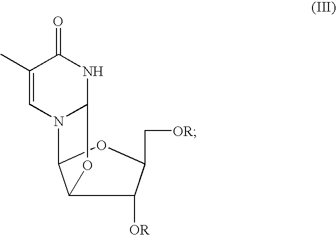
wherein the group R is as defined for formula (II);
c) a third step in which the product obtained in step b) is reacted with a nucleophile to cleave the C—O— bond in the 2′-position to obtain a thymidine derivative of formula (IV) having a reducible carbon in the 2′ position: 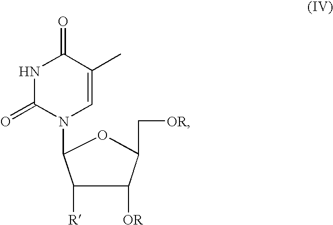
where R′ denotes the nucleophile radical and the group R is as defined for formula (II); and
d) a fourth step in which the product obtained in the third step c) is reductively converted into beta-L-2′deoxy-thymidine,
wherein optionally any protective groups that are present in compounds (III) or (IV) are cleaved during the third step, the fourth step, or in a subsequent step after the fourth step.

2. A process according to claim 1, wherein in step (a) the reaction with cyanamide is carried in the presence of a tertiary nitrogen base or an alkali or dialkali metal carbonate.

3. A process according to claim 1, wherein in step a) the hydroxy groups in the 3- and 5-positions of the L-arabinose are not reacted with a protective group.

4. A process according to claim 1, wherein in step a) the hydroxy groups in the 3- and 5-positions of the L-arabinose are reacted with a protective group.

5. A process according to claim 4, wherein in step a) the protective groups for the two hydroxy groups are selected from benzyl, diphenylmethyl, triphenylmethyl or silyl-protective groups, wherein the three substituents of the silyl protective group are selected from C₁-C₆-alkyls and/or phenyl, wherein the phenyl group may optionally be substituted with C₁-C₆-alkyl, nitro or C₁-C₆-alkoxy.

6. A process according to claim 4, wherein in step a) the protective groups are selected from trimethylsilyl, dimethyl-tert.butyl-silyl, diphenyl-tert.butyl-silyl or tributylsilyl protective groups.

7. A process according to claim 1, wherein in step b) the 2-methyl-C-3-acid or derivative thereof is selected from methyl-2-formyl-propionate, 2-formyl-propionitrile, a 2-formyl-propionic acid ester, 2-formyl-propionic acid azide, 2-formyl-propionic acid halide, a dimethoxy or diethoxy-acetal of the said formyl compounds, or a 3-z-2-methyl-2-propenooic acid ester, azide, halide or nitrile, wherein z is selected from F, Cl, Br, I, O-tosylate, or C₁-C₆-alkoxy.

8. A process according to claim 7, wherein the esters of said 2-formyl-propionic acid ester and 3-z-2-methyl-2-propenooic acid ester are selected from the methyl, ethyl, propyl or butyl-esters.

9. A process according to claim 7, wherein the 2-methyl-C-3-acid or derivative thereof is methyl-2-formylpropionate or 3,3-dimethoxy-2-methylpropionate.

10. A process according to claim 1, wherein in step b) a catalyst is used selected from tertiary nitrogen bases and inorganic salts.

11. A process according to claim 1, wherein in step b) a catalyst is used selected from dimethylaminopyridine, triethylamine, N-methylmorpholine, or mixtures thereof.

12. A process according to claim 1, wherein in step c) the nucleophile is selected from a Cl-anion, Br-anion, I-anion, tosylate or thioacetate, each of which is used in the form of the free hydrogen acids or salts thereof.

13. A process according to claim 12, wherein the nucleophile is selected from a Cl-anion, Br-anion, and an I-anion.

14. A process according to claim 12, wherein the nucleophile is a Br-anion.

15. A process according to claim 12, wherein the nucleophile is used in the form of the free hydrogen acids thereof.

16. A process according to claim 1, wherein step d) is carried out under a hydrogen atmosphere with a metal catalyst.

17. A process according to claim 18, wherein the metal catalyst is Raney nickel or palladium.

18. A process according to claim 1, wherein step d) is carried out in the presence of tributyltin hydride and a radical starter.

19. A process according to claim 1, consisting essentially of the steps set forth in claim 1.

20. A compound of Formula (II): 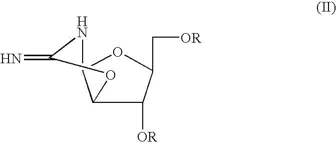
wherein R is hydrogen or a protective group.

21. A compound of Formula (III): 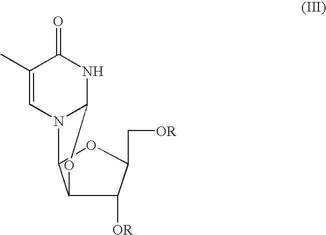
wherein R is hydrogen or a protective group.

22. A compound of Formula (IV): 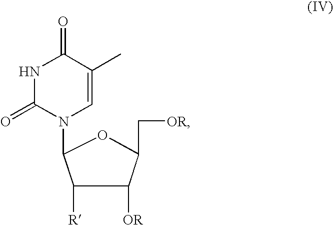
wherein R′ is a nucleophile radical and R is hydrogen or a protective group, provided that R′ is not thioacetate if R is methylbenzyl.

23. A compound of Formula (V): 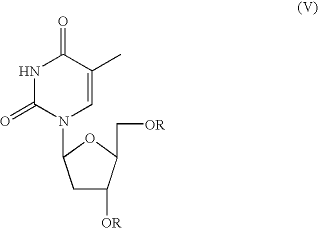
where R is trimethylsilyl or tributylsilyl.
